(19) United States
(12) Patent Application Publication
(10) Pub. No.: US 2013/0267601 A1
(43) Pub. Date: Oct. 10, 2013

(54) Method for prevention of alzheimer's disease

(71) Applicant: Joseph Michael Abramowitz, San Diego, CA (US)

(72) Inventor: Joseph Michael Abramowitz, San Diego, CA (US)

(21) Appl. No.: 13/506,286

(22) Filed: Apr. 9, 2012

(51) Int. Cl. A61K 31/197; A61P 25/28

(52) U.S. Cl. 514/567

(57) ABSTRACT

A method for prevention of Alzheimer's Disease (AD) is presented. The principal cause of AD is a deficiency at the cellular level of the human brain, of the effects of the active form of thyroid hormone, tri-iodothyronine (T3). Lifelong T3 replacement, beginning in early to mid life and, in some cases later, overcomes these blocks to T3 production and actions and prevents AD in a majority of the sub-types of AD.

### BACKGROUND TO THE INVENTION

### A: General Considerations:

The prevalence of AD has reached epidemic proportions as human life expectancy has increased. A link between thyroid hormone underactivity and AD has been postulated previously (1). Retrospective studies have demonstrated that treatment with thyroid hormones over decades significantly reduces the incidence of AD. Until now, no theoretical explanation has existed for these retrospective observations. The complexity of this entire issue is magnified by the following:

(i): A National Library of medicine search on the Phrase ‘thyroid hormone and Alzheimers disease’ received only 75 ‘hits’, attesting to the fact that this area of medical research has been all but ignored. (ii):Thyroid abnormalities may be mischaracterized in some studies; for example cases classified as borderline hyperthyroidism (overactive thyroid hormone activity) may actually be cases of secondary hypothyroidism (underactive thyroid hormone activity of central origin). (iii): Some studies measure TSH (thyroid stimulating hormone) and T4, but not T3, which is arguably the most critical of the thyroid hormones. (iv):Currently accepted normal ranges for the thyroid chemical moieties are incorrect and need to be revised. (v): In a medical academic culture obsessed with super-specialization, few are able to see the ‘big picture’.

(B): Review of the Evidence for a Relationship Between TH and AD: Dr. Broda Barnes followed a cohort of his patients treated for decades with thyroid hormone replacement and found that the incidence of AD in this cohort was dramatically lower than what would have been expected in the general population(1). It has been generally acknowledged by multiple sources that patients with diseases related to low thyroid hormone activity have an approximately two-fold increase in the incidence of AD.
(C): Comparison of Dementia Seen in Hypothyroidism with that Seen in AD:

Compared with AD, the cognitive decline seen with hypothyroidism (low thyroid hormone activity) is far less complex. Aside from the fact that diagnosis can be challenging when the free hormone levels are within the ‘normal’ range, the diagnosis of hypothyroid dementia should be relatively easy and should respond dramatically to thyroid hormone replacement in a few weeks, provided that the diagnosis is made early. This disease is different to AD, although there appears to be some overlap. The thyroid hormone abnormality that results in AD is far more chronic and insidious and results in a far greater burden of anatomic pathology in the brain.

### (D): Description of the Molecular Pathophysiology:

The key role of the thyroid gland is to produce iodinated forms of the amino acid L-tyrosine. The thyroid hormones are crucial for regulation of the metabolic rate (metabochrone function) of the human body and for the maintenance of the integrity of the function of a multitude of receptors located on the membranes of all cells in the human body (locksmith function). These thyroid hormones are named L-thyroxine (tetra-iodothyronine or T4) and tri-iodothyronine (T3). L-thyroxine contains four iodide substitutions. Tri-iodothyronine contains three iodide substitutions. The thyroid produces these hormones in a ratio of approximately T4:T3=15:1. T3 is approximately four to five times more potent than T4. it is generally believed that T3 is the active form of thyroid hormone. Following release from the thyroid gland, T3 and T4 circulate in the bloodstream. Approximately 99.9% of each is protein bound. An amount equal to less than 0.1% of each is free in the plasma, free to attach to thyroid hormone receptors to carry out the executive functions of the thyroid hormones. As T3 is the active form of the thyroid hormones, for proper functioning of thyroid hormone to occur, T4 must be de-iodinated in the thyroid or in the peripheral tissues by a group of enzymes, the iodothyronine deiodinases. Three forms of iodothyronine deiodinase have been identified. The most important for the conversion of T4 to T3 is believed to be iodotyrosine deiodinase type 2.

### The Iodotyrosine Diodinases (DI):

1. DI 1: Found in the liver, kidney, thyroid and pituitary. Bound to the plasma membrane (the membrane surrounding the cell).
2. DI 2: Found in the heart, skeletal muscle, fat, pituitary, central nervous system and thyroid. Bound to the membrane of the endoplasmic reticulum (an intracellular organelle involved with protein synthesis).
3. DI 3: Found in brain, fetal tissue and in the placenta.

The deiodinases convert T4 to T3 in the thyroid and in all peripheral tissues by deiodinating the outer ring of the prohormone (T4) to produce T3, the active hormone. It is believed that the deiodinases are responsible for maintaining the appropriate ratio of T4/T3 in every tissue in the body. It should be readily apparent from the foregoing that subnormal activity of DI's will result in lower levels of circulating and intracellular T3. As T3 is one of the most important hormones in the body, the effects of subnormal levels of T3 may be significant, if not devastating.

### (E): The Missing Pieces of the Puzzle:

Only a sufficient quantity of tri-iodothyronine present consistently throughout the life of a human can prevent the biochemical and subsequent neuropathologic changes leading to the development of dementia of the Alzheimer type in genetically susceptible individuals. Tri-iodothyronine serves this protective function via it's effects on lipoprotein and lipoprotein receptor metabolism, regulating and maintaining normalcy of cholesterol and overall lipid metabolism in the brain. To this end, tri-iodothyronine also functions in other tissues, specifically (but not exclusively) the liver, modulating lipoprotein metabolism and lipid clearance. In regard to prevention of AD, the most crucial role of this physiologic homeostatic mechanism is the clearance of amyloid beta peptide (essentially a central nervous system waste product) from the human brain. In the absence normal T3 activity, amyloid beta peptide accumulates in the brain in the form of amyloid plaques, contributing to the pathophysiology and anatomic pathology of AD. Decreases in sex hormone activity also play a contributory role, although the role and its' significance in men is uncertain. The critical effect of sex hormone deficiency after the female menopause, coupled with the fact that thyroid disease is more common in women, account for the observation that AD is approximately twice as common in women as in men. In a genetically susceptible patient approaching late middle age, the brain is unable to generate adequate quantities of intracellular T3, via qualitatively or quantitatively defective intracellular T3 activity, or via other related biochemical aberrations. The brain is unable to clear excessive quantities of amyloid beta peptide, which accumulates, setting the stage for progression to the anatomic pathology of AD and the subsequent clinical manifestations.

(F): Why Has this New Art Been Missed by Mainstream Medicine?
1. TH physiology taught to medical students and doctors is incorrect.
2. Normal reference ranges for thyroid laboratory parameters, at the time of development of this new art, were incorrect.
3. Many hypothyroid patients are misdiagnosed by their doctors as having normal thyroid function. This phenomenon currently constitutes the cause, and cryptic nature, of the most spectacular and large-scale epidemic in the history of the human species.
4. Peri- and post-menopausal females are discouraged against taking estrogen replacement therapy, even when they are at low risk of developing the cardiovascular and oncogenic complications of such treatment. 5. Development of studies evaluating prevention of AD by the method described herein has been ignored because it is simple, cheap and not generative of the huge profits sought after by medical researchers and the pharmaceutical industry. 6. Most patients treated with thyroid hormone replacement are treated with T4. In patients unable to convert T4 to the active T3 (because they possess genetic defects in DI type 2) T4 administration will not prevent the intracellular starvation of the active TH (T3) effect and the subsequent progression to AD.

### (G): The Net Effects of the Above Confusion in Clinical Practice is:

Patients are misdiagnosed as euthyroid (having normal thyroid function) when they actually have a paucity of intracellular thyroid hormone effect. Patients who are treated with T4 alone and who are unable to convert T4 to T3 remain hypothyroid at the cellular level.

### SUMMARY OF THE INVENTION

Lifelong administration of exogenous T3 prevents the molecular biologic, neuropathologic and clinical consequences of diminished brain thyroid hormone activity, which manifests in genetically susceptible patients as AD. The method of the invention described herein involves administration of exogenous T3, beginning around the time of the andropause (male menopause) or gynopause (female menopause) generally, but not exclusively, prior to age 50.

### DETAILED DESCRIPTION OF THE DIAGRAMS

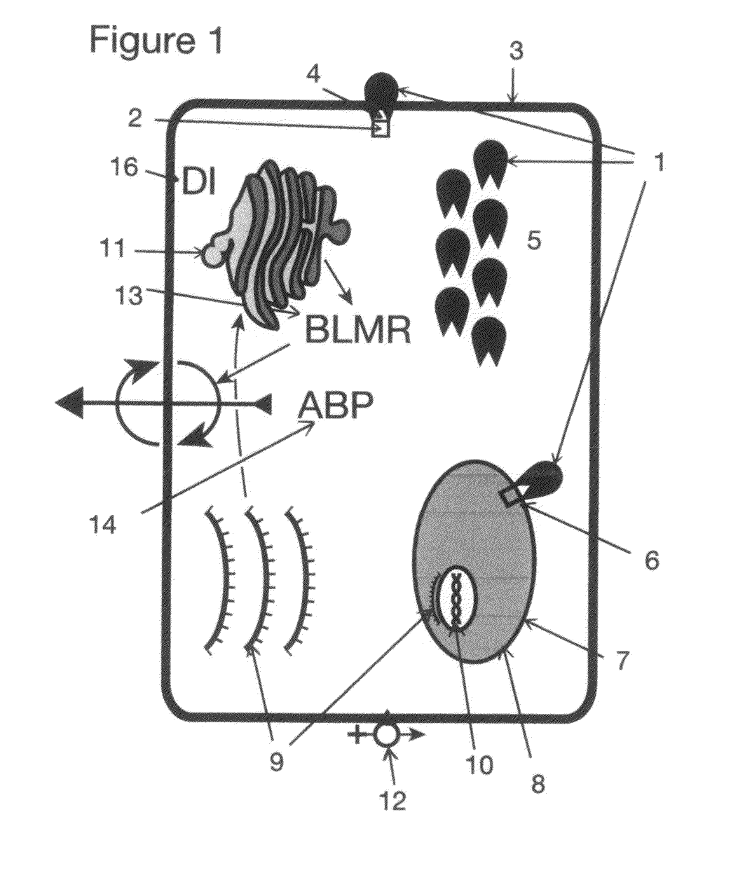

### FIG. 1: Applied Clinical Pharmacology and Normal Physiology:

What follows is a description of the art of the present invention applied to an afflicted human, destined to develop AD, by application of the preventative treatment art embodied in the claims described hereinunder, which is materially the same as the normal physiologic processes in an unafflicted human. Thyroid hormone receptor agonist 1 docks with the thyroid hormone receptor 2 on the surface of the human brain cell body 3. Thyroid hormone membrane transporter 4 carries the thyroid hormone receptor agonist into the interior of the cell. Via this transport process (and, in the case of T4, deiodination by the enzyme iodothyronine deiodinase, DI 16), T3 is now in the cytoplasm 5, in the interior of the cell. T3 then passes through the cytoplasm to dock with the thyroid hormone receptor 6 on the nuclear membrane 7 of the cell nucleus 8. Inside the nucleus, T3 activates the transcription process whereby multiple messenger RNA (m-RNA) 9 molecules are generated from the DNA 10 of the cell, which code for a host of critical regulator proteins. These m-RNA molecules then pass out of the nucleus and into the cytoplasm, where they are directed to the endoplasmic reticulum 11, a cytoplasmic organelle which functions to translate the m-RNA 9 into the critical regulator proteins themselves. In settings in which insufficient intracellular T3 is present to activate the DNA transcription cascade, sex hormones 12, if present in sufficient concentration, may play a surrogate role taking over or supplementing the T3 effects. Once produced, the regulator proteins proceed to their loci of function in other parts of the cell. One group of regulator proteins, termed brain lipid master regulators (BLMR) 13 is responsible for normal brain lipid homeostasis, regulating and maintaining intermediary brain lipid metabolism, including that of lipid substrates and lipoproteins and their respective receptors on the cell membrane on the exterior of the cell. Through as yet incompletely understood mechanisms, which include salutary effects of T3 dependent liver lipoprotein metabolism via liver (hepatic) lipid master regulators (HLMR), T3 dependent lipid regulation in the brain and in the liver cooperate to facilitate the clearance of amyloid beta peptide (ABP) 14 from the brain. Consequently ABP does not accumulate in the brain cell and amyloid plaques do not develop. The molecular pathophysiology of AD does not develop and the clinical disease known as dementia of the Alzheimer type is prevented.

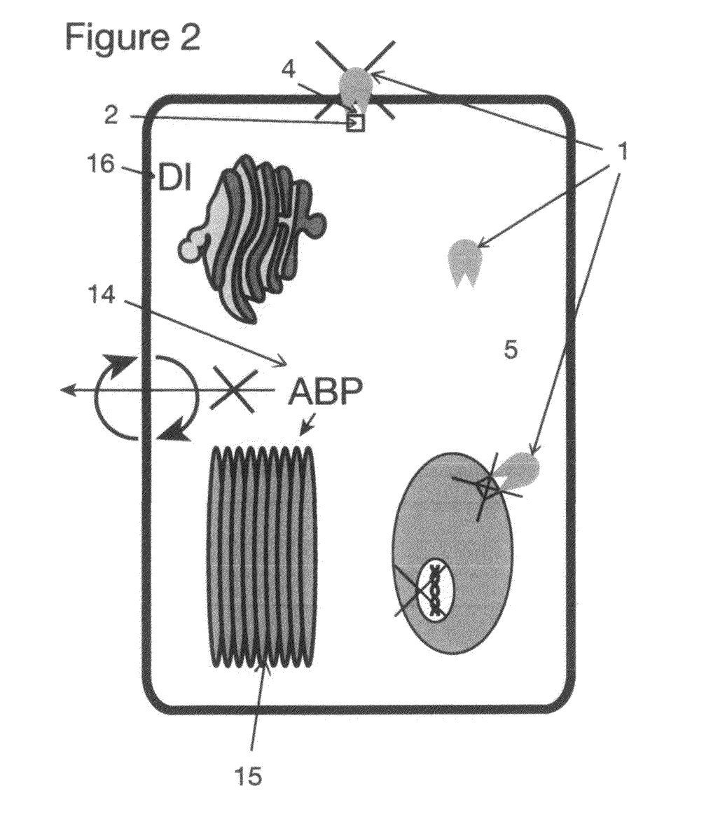

### FIG. 2: Molecular Pathophysiology of Alzheimers Disease:

What follows is a description of the derangements in the normal physiology described in diagram 1, in a human with a genetic susceptibility for the development of AD, in whom an adequate sufficiency of T3 in the cytoplasm of the human brain cell is absent. Cytoplasmic T3 1 exists in the cytoplasm 5 in a concentration below the threshold necessary for carrying out the critical function of inducing the production of BLMR. This may occur via multiple mechanisms. Thyroid hormone receptor agonists 1 may not be present in sufficient concentration outside the cell for the development of a sufficient concentration of T3 1 inside the cell. Alternatively the thyroid hormone receptor 2 or the membrane transporter 4 may be defective. Alternatively, membrane and/or intracellular iodothyronine deiodinase 16 may be defective. In the preferred embodiment of the invention, the chief consequence of the aforementioned defects (diminished concentration of intracellular T3), individually or in combination, can be overcome by the exogenous administration of thyroid hormone receptor agonists as described in the claims below. The liver of this index afflicted human is also exposed to subthreshold quantities of T3, such that T3 dependent hepatic processes are also impaired. Consequently the production of HLMR does not occur. When the liver-brain cooperative ABP 14 clearance mechanism functions at this subthreshold level in regard to these critical executive functions of T3, the transport of ABP 14 out of the cell is retarded and ABP 14 builds up in the cell, forming amyloid plaques 15 (AP). It is not known whether single or multiple blocks to ABP 14 clearance exist. In the former case, it is not known whether the block is in the brain cell, liver cell or elsewhere. The buildup of amyloid plaques 15 and/or the continued paucity of intracellular T3 1 effect, progressively cripple the energy generating processes within the cell. Another critical function of thyroid hormone is the maintenance of the metabolic rate in every cell in the body, hereafter known as the ‘metabochrone effect’. The combined effect of the aforementioned results in a death-knell positive feedback effect which accelerates the vicious cycle of brain cell dysfunction, degeneration and death. This phenomenon may well account for periods of rapid clinical deterioration frequently seen in patients with established Alzheimers disease. This occurs chiefly because the thyroid hormone membrane transporter 4 requires energy in order to function. Intracellular power plant failure, a consequence of brain cell dysfunction, starves the membrane transporter of needed energy such that it becomes sluggish, further diminishing the concentration of T3 1 in the cytoplasm of the cell. The chronic buildup of AP 15 in the human brain cell eventually results in a sufficient burden of molecular pathophysiology so as to impede normal brain function leading to the clinical disease known as dementia of the Alzheimer type.

What is claimed is:

1. A method for prevention of Alzheimer's disease, in a human, comprising ensuring that an adequate sufficiency of tri-iodothyronine is present inside the cells of the human brain, throughout the life of the human, whereby the patho-physiology and patho-anatomy of Alzheimer's disease is prevented, preventing the clinical disease of Alzheimer's disease.

2. The method of claim 1, whereby the prevention of Alzheimer's disease is achieved by the exogenous administration of tri-iodothyronine.

3. The method of claim 1, whereby the prevention of Alzheimer's disease is achieved by the exogenous administration of levo-thyroxine.

4. The method of claim 1, whereby the prevention of Alzheimer's disease is achieved by the exogenous administration of a substance which is an agonist at the human thyroid hormone receptors, but which is not a hormone produced by the thyroid gland.

5. The method of claim 1, whereby the prevention of Alzheimer's disease is achieved by the exogenous administration of a substance which is not a direct thyroid hormone receptor agonist, but which modifies intermediary metabolism of the thyroid hormones produced by the body of the human or modifies second messenger functions of thyroid hormone, such that a molecular biologic defect, peculiar to that human and leading to blocks in thyroid hormone function in the brain, and leading to Alzheimer's Disease, is overcome.

6. A method for prevention of Alzheimer's disease, in a perk or post-gynopause (menopause) female human comprising ensuring, via exogenous administration, that an adequate sufficiency of members of the class of estrogen hormones is present and available for physiologic function in the human brain.

7. The method of claim 6, which combines necessary elements of claims 1-5.

8. A method for prevention of Alzheimer's disease, in a perk or post-andropause male human comprising ensuring, via exogenous administration, that an adequate sufficiency of the class of testosterone hormones is present and available for physiologic function in the human brain.

9. The method of claim 8, which combines necessary elements of claims 1-5.
